(19) United States
(12) Patent Application Publication
(10) Pub. No.: US 2023/0313128 A1
(43) Pub. Date: Oct. 5, 2023

(54) METHODS FOR IMPROVED MICROSPORE EMBRYOGENESIS AND PRODUCTION OF DOUBLED HAPLOID MICROSPORE-DERIVED EMBRYOS

(71) Applicant: Seminis Vegetable Seeds, Inc., St. Louis, MO (US)

(72) Inventor: Stefano Pessina, Roquetas de Mar (ES)

(21) Appl. No.: 18/189,935

(22) Filed: Mar. 24, 2023

Related U.S. Application Data
(60) Provisional application No. 63/326,011, filed on Mar. 31, 2022.

(51) Int. Cl. C12N 5/04; A01H 1/08

(52) U.S. Cl. CPC C12N 5/04; A01H 1/08; C12N 2500/34; C12N 2500/05; C12N 2500/46; C12N 2500/32

(57) ABSTRACT

The invention provides novel methods and compositions for microspore embryogenesis and the production of doubled haploid embryos. For example, the methods provided include the steps of sterilization of pepper buds, microspore isolation from sterilized buds, liquid culture of microspores, a double-layer subculture, embryo harvest for germination and conversion to plantlets, and acclimatization of cultured plantlets.

### CROSS-REFERENCE TO RELATED APPLICATIONS

This application claims the benefit of U.S. Provisional Appl. Ser. No. 63/326,011, filed Mar. 31, 2022, the disclosure of which is incorporated herein by reference in its entirety.

### FIELD OF THE INVENTION

The present invention relates to the field of plant breeding and agricultural biotechnology. More specifically, the invention provides methods for pepper microspore embryogenesis and doubled haploid production.

### BACKGROUND OF THE INVENTION

The use of doubled haploids (DH) in a plant breeding program allows new inbred populations to be created from desired parents in 1-2 generations. Haploids (in) contain only one set of chromosomes that are found after meiosis in male or female gametes. Doubled haploids (2n) carry two identical sets of chromosomes that were derived from haploids. Unlike ordinary diploids that achieve 2n chromosomes through fertilization between male and female gametes, DH become diploid through chromosome doubling of the haploid chromosome by chemical or spontaneous means. Utilization of DH can enable breeding to obtain pure 2n homozygous plants in a single generation, compared to 6 or more generations of selfing or backcrossing in typical breeding schemes. It is a critical tool to reduce breeding cycle time with improved heritability to accelerate genetic gain. In addition, DH is also a useful tool to obtain homozygous plants faster in other processes, such as gene stacking, genome editing, cytoplasm and nuclear genome exchange, trait integration, etc. (Ren et al. 2017).

Although DH technologies have been developed successfully in rapeseed, barley, and other Solanaceae like tobacco, pepper is known for been a challenging crop for androgenesis induction. Many factors influence the production of pepper DH, for example: genotype of donor plant, selection of starting material, stress induction treatment, culture environment and cultivation method. The genotype of a donor plant greatly influences the responsiveness of gametophytes to the in vitro culture conditions, in addition several pepper varieties behave recalcitrant to the in vitro androgenesis. Selection of the pepper bud also has also an effect in the success or the technique, different morphological markers (bud length, pigmentation, calyx/bud ratio) can be used to predict developmental stage (Parra-Vega et al. 2013). Exposing the gametes to stressful conditions, such as heat shock and sucrose-starvation, promote embryogenesis in hot pepper (Kim et al. 2008). Additionally, to the stress induction cultivation, pepper microspores can be treated with chemical compound (e.g. with Trichostatin A) that acts as an epigenetic regulator by blocking histone deacetylase (HDACs) and increasing embryogenic cell divisions in C. annuum (EP 3048874 B 1, EP 3328185 B1). Another factor to consider is the culture environment for both microspore induction and the embryo regeneration. A two-step culture method for microspores showed that combining a liquid cultivation followed by a double layer medium cultivation, improves the efficiency of a microspore-derived embryos obtained (Kim et al. 2013). Although the examples mentioned previously represent useful optimization steps towards improved pepper DH production, according to Seguí-Simarro 2016, “the problem of embryo quality and ability to germinate still appears as a major bottleneck to be overcome”.

For microspore culture-based DH to be of value to a breeding program, the most important metric is the efficiency of embryogenesis. However, improving embryogenesis efficiency alone does not necessarily lead to an increase in doubled haploids. While there are many known processes that increase embryogenesis efficiency, these processes are of little or no value to a breeding program if only a very small percentage of the embryos have doubled chromosomes. The present inventors have developed a novel method, which is described herein, that significantly improved the germination of embryos and enhanced the production of pepper DH. Using this invention leads to a fivefold increase in the amount of DH obtained per pepper bud when compared against a conventional anther culture method. Furthermore, this invention has been tested in more than 110 different pepper origins, including both sweet and hot pepper varieties. Surprisingly, the responsiveness of recalcitrant material was increase 10-fold by employing the new microspore method. This serves to deliver significant time and resource savings.

### SUMMARY OF THE INVENTION

In one aspect, the present invention provides a method for producing embryos from microspores comprising the steps of: a) isolating microspores from flower buds obtained from a donor pepper plant, wherein the microspores are at a developmental stage competent for induction of embryo development and wherein said microspores are isolated in a washing medium; b) applying a stress treatment to said microspores comprised in an induction medium; c) culturing said treated microspores in a culture medium; d) subculturing said treated microspores as the liquid phase of a double layer culture medium; and e) harvesting said embryos on a solid medium suitable for germination, wherein said medium comprises an auxin, a gibberellin, and a cytokinin, and wherein said cytokinin is present in said germination medium at a ratio of about 1:1 to about 10:1 relative to said auxin. In some embodiments, the washing medium comprises Gamborg's B5 basal salts, Gamborg's B5 vitamins, and an organic carbon source, wherein said organic carbon source is present in said medium at a high concentration. In other embodiments, the organic carbon source is present in said washing medium at a concentration of about 50.0 g/L to about 200.0 g/L. In some embodiments, the organic carbon source is present in said washing medium at a concentration of about 90.0 g/L to about 130.0 g/L. In further embodiments, said organic carbon source is sucrose or maltose. In yet further embodiments, said organic carbon source is sucrose. In some embodiments, said stress treatment comprises incubating said microspores at a temperature of about 31° C. to about 33° C. for about 24 hours to about 96 hours. In other embodiments, said stress treatment is carried out on microspores present in said medium at a density of about 6.0×10⁴ cells/mL to about 10.0×10⁴ cells/mL. In some embodiments, said stress treatment is carried out on microspores present in said medium at a density of about 6.80×10⁴ cells/mL to about 9.20×10⁴ cells/mL. In other embodiments, said culturing is carried out at a density of about 3.75×10⁴ cells/mL to about 6.25×10⁴ cells/mL. In further embodiments, said culturing is carried out at a density of about 4.25×10⁴ cells/mL to about 5.75×10⁴ cells/mL. In other embodiments, said subculturing is carried out at a density of about 1.125×10⁴ cells/mL to about 1.875×10⁴ cells/mL. In additional embodiments, said subculturing is carried out at a density of about 1.28×10⁴ cells/mL to about 1.73×10⁴ cells/mL. In some embodiments, harvesting comprises harvesting the embryos on at least one filter paper on top of said solid medium. In other embodiments, said germination medium comprises pyridoxine HCl present in said medium at a concentration of about 0.05 mg/L to about 5.0 mg/L.

In another aspect, the present invention provides a method for producing embryos from microspores comprising the steps of: a) isolating microspores from flower buds obtained from a donor pepper plant, wherein the microspores are at a developmental stage competent for induction of embryo development and wherein said microspores are isolated in a washing medium; b) applying a stress treatment to said microspores comprised in an induction medium; c) culturing said treated microspores in a culture medium; d) subculturing said treated microspores as the liquid phase of a double layer culture medium; and e) harvesting said embryos on a solid medium suitable for germination, wherein said medium comprises an auxin, a gibberellin, and a cytokinin, and wherein said cytokinin is present in said germination medium at a ratio of about 1:1 to about 10:1 relative to said auxin, and wherein said auxin is selected from the group consisting of: 1-naphthaleneacetic acid, indole-3-acetic acid, and indole-3-butyric acid. In some embodiments, said auxin is indole-3-acetic acid. In other embodiments, said cytokinin is kinetin or thidiazuron. In further embodiments, said cytokinin is thidiazuron. In some embodiments, said gibberellin is gibberellic acid. In other embodiments, said cytokinin to auxin ratio is from about 2:1 to about 5:1. In further embodiments, said cytokinin to auxin ratio is about 4:1. In other embodiments, said gibberellin is present in said germination medium at a concentration of about 0.01 mg/L to about 10 mg/L. In some embodiments, said cytokinin is present in said germination medium at a concentration of about 0.1 mg/L to about 10 mg/L. In other embodiments, said auxin is present in said germination medium at a concentration of about 0.1 mg/L to about 10 mg/L.

In yet another aspect, the present invention provides a method for producing embryos from microspores comprising the steps of: a) isolating microspores from flower buds obtained from a donor pepper plant, wherein the microspores are at a developmental stage competent for induction of embryo development and wherein said microspores are isolated in a washing medium; b) applying a stress treatment to said microspores comprised in an induction medium; c) culturing said treated microspores in a culture medium; d) subculturing said treated microspores as the liquid phase of a double layer culture medium; and e) harvesting said embryos on a solid medium suitable for germination, wherein said medium comprises an auxin, a gibberellin, and a cytokinin, and wherein said cytokinin is present in said germination medium at a ratio of about 1:1 to about 10:1 relative to said auxin, and wherein said germination medium further comprises activated charcoal, polyvinylpyrrolidone, leucine, or spermidine. In some embodiments, said activated charcoal is present in said germination medium at a concentration of about 0.1 mg/L to about 10 mg/L. In other embodiments, said polyvinylpyrrolidone is present in said germination medium at a concentration of about 0.5 mg/L to about 5.0 mg/L. In some embodiments, said leucine is present in said germination medium at a concentration of about 1.2 mg/L to about 120.0 mg/L. In other embodiments, said spermidine is present in said germination medium at a concentration of about 1.4 mg/L to about 140.0 mg/L. In further embodiments, said germination medium further comprises activated charcoal, polyvinylpyrrolidone, leucine, and spermidine.

### BRIEF DESCRIPTION OF THE DRAWINGS

FIG. 1: Shows an efficiency comparison (DH/bud) using various formulations of germination media compared to the traditional anther germination medium.

FIG. 2: Shows an efficiency comparison (DH/bud) between the anther culture protocol vs the new microspore culture protocol method across different pepper types tested.

FIG. 3: Shows an efficiency comparison between anther culture and microspore culture using recalcitrant pepper genotypes. There was an increase in responsiveness of recalcitrant pepper genotypes when using the new method for microspore induction and culture.

### DETAILED DESCRIPTION

Doubled haploid (DH) plants are a valuable tool to plant breeders, particularly for generating inbred lines. A great deal of time is spared as homozygous lines are essentially generated in a single generation, negating the need for multigenerational conventional inbreeding, thus accelerating breeding genetic gain. In addition, DH plants are entirely homozygous, thus there is no allele masking effect between genotype and phenotype. They are very amenable to breeding selection and quantitative genetics studies. For breeders, DH populations have been particularly useful in QTL mapping, cytoplasmic conversions, and trait introgression.

DH plants have traditionally been obtained through the use of gametophytes of female and male origin using in vitro approaches to divert them from their normal developmental pathway of becoming functional gametes or accessory cells to instead become embryos in the absence of fertilization. This is achieved by exploiting the totipotency capacity of gametophytic cells through use of stress-inducing conditions that avoid cell death and instead reprogram the haploid cell to enter an embryogenic pathway. The production of haploid/DH plants from female gametophytes is commonly known as induction of in vitro gynogenesis whereas in vitro androgenesis is the generation of male-derived progeny. In both techniques, the resultant regenerated embryo will contain the same genetic background of the donor plant from which the gametophytes were obtained.

Pepper is a vegetable cultivated worldwide and a key ingredient in many food cultures. Therefore, breeding programs must adapt their technologies to develop new commercial hybrids of sweet and hot pepper varieties. To achieve that goal, an efficient in vitro culture protocol is required to accelerate DH production by improving the regeneration of embryos into DH plants. In pepper, DH production based on androgenesis has been most successful using either anthers or microspores (immature pollen) as the starting material. Of the two, anther culture is the more widely used method as it has the advantages of being relatively simple and inexpensive. The basic steps of anther culture are: flower bud collection, isolation of anthers from buds, inoculation of anthers on agar-based culture medium, isolation of embryos, regeneration of plants, and analysis of regenerants. Due to its widespread use over the last several decades, this method has been modified and developed extensively to be successfully applied to many different breeding programs. However, one major drawback to this method is its low efficiency due to the small quantity of embryos that are typically obtained from each anther. This is difficult to overcome as obtaining a large number of anthers is laborious and requires high level of expertise and thus creates a bottleneck in automating and scaling up the number of DH plants obtained per flower bud using this method for crop breeding. Furthermore, since anther culture involves culture of whole anthers, regenerated plants produced by this method may not exclusively be derived from microspores but also from somatic tissue since embryoids or callus can develop not only from microspores, but also from somatic tissues of the anther.

Isolated microspore culture does not have the risk of regenerated plants being of different origins as this method requires isolation of microspores from the anthers prior to culture. This avoids inadvertent formation of callus and embryos from somatic tissue and all regenerated plants are presumably haploids or double haploids derived from microspores. Using isolated microspores also enables better control of medium composition during culture, since no other tissues are present to potentially modify the medium composition by the secretion of beneficial or harmful compounds. Isolated microspore culture is also advantageous due to a higher efficiency of embryo production, since microspores have better accessibility to nutrients in liquid culture and developing embryos are not limited by the reduced space of the anther locule. Isolated microspore culture also has its disadvantages, especially in its use in pepper breeding. For example, specific culture medium formulations are required for various steps in the protocol due to the absence of anther tissue which, under normal circumstances, supports proper microspore growth and development. Furthermore, microspores of different species and cultivars within a species can have much different requirements for embryogenic development and protocols must be tailored based on these differences. Because microspore culture for the production of DH plants is newer and less commonly applied to breeding programs, there is a great need to optimize the steps in the process as they specifically relate to pepper production.

### Embryogenesis and Doubled Haploids

Microspore-derived embryogenesis is a unique process in which haploid, immature pollen (microspores) are induced by one or more stress treatments to form embryos in culture. There is no single standard condition or protocol for obtaining plant formation by isolated microspore culture. Microspores of different species and cultivars within a species can have much different requirements for embryogenic development. The medium composition and the genotype response are among the key factors for a successful DH production system of microspore culture. Exemplary methods of microspore culture are disclosed in, for example, U.S. Pat. Nos. 5,322,789 and 5,445,961, the disclosures of which are specifically incorporated herein by reference. The present invention provides a novel protocol for culture of pepper microspores for the production of doubled haploid plants, including the steps of sterilization of pepper buds, microspore isolation from sterilized buds, liquid culture of microspores, a double-layer subculture, embryo harvest for germination and conversion to plantlets, and acclimatization of cultured plantlets. Microspores can be isolated from plants by crushing surface-sterilized flower buds under conditions which divert the microspores from gametophytic development to that of embryogenic development, such as in an isolation medium capable of maintaining microspore viability and embryogenic potential. These isolated microspores are then exposed to an embryoid/callus promoting medium. Responsiveness in the microspore culture depends on the genotype.

Regeneration of microspore embryos into normal plants is an important process for applied DH production. Unlike zygotic embryos, microspore embryos can germinate into plantlets without having gone through the typical maturation processes that prepare the zygotic embryo for dormancy. Nonetheless, the frequency of plantlet conversion is low in some species. The conversion efficiency depends on the developmental stage of embryo, the germination medium and other culture conditions.

A bottleneck in the current doubled haploid production protocols used with pepper is the low germination success of the embryos generated from isolated microspore culture. The conversion efficiency depends on the germination medium and other culture conditions. Depending on the developmental stage, tissue culture can have different nutritional and phytohormone requirements. Until recently, androgenesis induction protocols employed basal medium recipes that were developed almost 60 years ago and were extensively used for a wide range of in vitro culture applications. For example, MS medium (Murashige & Skoog, 1962) was developed to promote organogenesis of tobacco tissue culture; similarly, B5 medium (Gamborg et al., 1968) was developed to supply the nutrient requirements of soybean roots during in vitro culture. The present inventors developed an improved method for pepper microspore embryogenesis, which focuses on improving the germination rates of microspore-derived embryos. Notably, a novel germination medium was developed by testing various combinations of compounds, such as micronutrients, macronutrients, vitamins, amino acids, growth regulators, antioxidants, and polyamines as supplements to the basal MS medium recipe, until key components and their concentrations were identified as playing a role in successful embryo germination. It was found that the methods and compositions described herein improved germination rates over a variety of pepper genotypes when compared to the germination rates using standard methods of microspore culture.

As used herein, the term “tissue culture” indicates a composition comprising isolated cells of the same or a different type or a collection of such cells organized into parts of a plant. Exemplary types of tissue cultures are protoplasts, calli, and plant cells that are intact in plants or parts of plants, such as embryos, pollen, flowers, leaves, roots, root tips, anthers, and the like. In a preferred embodiment, the tissue culture comprises microspores, embryos, protoplasts, meristematic cells, pollen, leaves or anthers derived from immature tissues of these plant parts. Means for preparing and maintaining plant tissue cultures are well known in the art (U.S. Pat. Nos. 5,538,880 and 5,550,318, each incorporated herein by reference in their entirety). By way of example, a tissue culture comprising organs such as anthers has been used to produce regenerated plants (U.S. Pat. Nos. 5,445,961 and 5,322,789; the disclosures of which are incorporated herein by reference).

As used herein the term “tissue culture media” refers to liquid, semi-solid, or solid media used to support plant growth and development in a non-soil environment. These tissue culture media can either be purchased as a commercial preparation or custom prepared and modified by those of skill in the art. Examples of such media include, but are not limited to those described by Murashige and Skoog (1962); Chu et al. (1975); Linsmaier and Skoog (1965); Uchimiya and Murashige (1962); Gamborg et al. (1968); Duncan et al. (1985); McCown and Lloyd (1981); Nitsch and Nitsch (1969); and Schenk and Hildebrandt (1972), or derivations of these media supplemented accordingly. Those of skill in the art are aware that media and media supplements, such as nutrients and plant growth regulators are usually optimized for the particular target crop or variety of interest. Other media additives can include but are not limited to amino acids, macroelements, iron, microelements, inositol, vitamins and organics, carbohydrates, undefined media components such as casein hydrolysates, with or without an appropriate gelling agent if desired for preparing semi-solid or solid medium. These tissue culture media may be used to prepare induction, washing, germination, plantlet development, or regeneration media.

In some embodiments, the methods described herein comprise isolating microspores from flower buds obtained from a donor pepper plant, wherein the microspores are at a developmental stage competent for induction of embryo development and wherein said microspores are isolated in a washing medium. A washing medium may comprise a variety of standard culture media or solution ingredients or components, such as for example, basal salts, macronutrients, micronutrients, a carbon source, antibiotics, and/or vitamins. The washing medium described herein comprises a basal salt medium, preferably Gamborg's B5 (Gamborg, 1968), supplemented with vitamins (specifically myo-inositol, nicotinic acid, pyridoxine HCl, and thiamine HCl ), sucrose or maltose, and a suitable antibiotic, such as cefotaxime. In specific embodiments, the washing medium further comprises polyvinylpyrrolidone.

According to many embodiments, the washing medium comprises a high concentration of an organic carbon source. As used herein “high concentration of an organic carbon source” refers to a medium comprising a total concentration of an organic carbon source, such as sucrose or maltose, that is greater than or equal to about 50.0 g/L. As used herein a “organic carbon source” refers to an exogenous organic component of plant tissue culture nutrient medium that facilitates in vitro growth and development by being the primary source of energy for cells. In plant cell culture, sugars are typically used as the organic carbon source. While sucrose is the most commonly used sugar, due to its high water solubility and electrical neutrality, maltose may also be used.

The washing medium may comprise, in some embodiments, a total organic carbon source concentration of greater than or equal to about 50.0 g/L, about 55.0 g/L, about 60.0 g/L, about 65.0 g/L, about 70.0 g/L, about 75.0 g/L, about 80.0 g/L, about 85.0 g/L, about 90.0 g/L, about 95.0 g/L, about 100.0 g/L, about 110.0 g/L, about 120.0 g/L, about 130.0 g/L, about 140.0 g/L, about 150.0 g/L, about 160.0 g/L, about 170.0 g/L, about 180.0 g/L, about 190.0 g/L, or about 200.0 g/L, including all concentrations derivable therebetween.

In some embodiments, the concentration of the organic carbon source in the washing medium is in the range from about 50.0 g/L to about 200.0 g/L, preferably from about 90.0 g/L to about 130.0 g/L, including all ranges derivable therebetween. A concentration of the organic carbon source that is considered high is more than 5% (w/v) to 20% (w/v) (200 g/L).

In some embodiments, the methods described herein comprise applying a stress treatment to isolated microspores comprised in a induction medium to induce embryogenesis and subsequent culturing and further subculturing of induced microspores for embryo development. A medium suitable for microspore culture, such as NLN medium (Lichter, 1982; Nitsch and Nitsch, 1967), is the base medium used in each of these steps and may be in liquid, semi-solid, or solid form, depending on the step. A semi-solid/solid medium may comprise a gelling or polymeric agent or ingredient that can solidify and form the medium. Agar, agarose, and gellan gums (Gelrite and Phytagel) are the most frequently used gelling agents in plant tissue culture. According to present embodiments, the induction medium comprises NLN liquid medium, supplemented with NLN salts and vitamins, gum arabic, cefotaxime, and mannitol. In some embodiments, induced microspores are cultured in NLN liquid medium supplemented with NLN salts and vitamins, gum arabic, cefotaxime, polyvinylpyrrolidone, and an organic carbon source, preferably sucrose. In other embodiments, cultured induced microspores are further subcultured using a double-layer culture system. In this system, the top layer is comprised of cultured induced microspores in NLN liquid medium supplemented with NLN salts and vitamins, gum arabic, cefotaxime, polyvinylpyrrolidone, and an organic carbon source, preferably sucrose, and the bottom layer is the same NLN formulation (NLN liquid medium supplemented with NLN salts and vitamins, gum arabic, cefotaxime, polyvinylpyrrolidone, and an organic carbon source, preferably sucrose) with the additional component of Gelrite/Phytagel to form a solid base.

In some embodiments, the methods described herein comprise harvesting embryos on a solid medium suitable for germination. Specifically, an embryo suspension is placed on top of at least one filter paper (preferably two or three filter papers) which are on top of a solid germination medium and are incubated at about 26° C. in a 16 hour light/8 hour dark photoperiod until embryos germinate. As used herein “germination medium” refers to a medium comprising a source of nutrients, such as vitamins, minerals, carbon and energy sources, and other beneficial compounds that facilitate the development of embryos into plantlets. According to present embodiments, a germination medium may comprise a variety of standard culture media or solution ingredients or components, such as for example, basal salts, macronutrients, micronutrients, sugars, antibiotics and/or vitamins. The germination medium for use according to the methods described herein may be described, in some embodiments, in terms of its composition of plant growth regulators, polyvinylpyrrolidone, polyamines, activated charcoal, and pyridoxine HCl.

In preferred embodiments, the germination medium comprises three different plant growth regulators, specifically an auxin, a gibberellin, and a cytokinin. Non-limiting examples of cytokinins that may be used in the accordance with the present disclosure may include, but are not limited to: 6-benzylaminopurine (BAP), thidiazuron (TDZ), kinetin, zeatin, N-(2-chloro-4-pyridyl)-N-phenylurea (4-CPPU), diphenyl urea (DPU), 6-(gamma,gamma-dimethylallylamino)purine (2iP), and 6-(3-hydroxybenzylamino)purine (meta-topolin). Auxins which may be used in accordance with the present disclosure may include, but are not limited to: indole-3-acetic acid (IAA), indole-3-butyric acid (IBA), naphthalene acetic acid (NAA), 2,4-dichlorophenoxy-acetic acid (2,4-D), 4-amino-3,5,6-trichloro-picolinic acid (picloram), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). A non-limiting example of a gibberellin that may be used in the accordance with the present disclosure is gibberellic acid (also called gibberellin A3, GA, and GA₃).

In preferred embodiments, the level of cytokinin activity is relatively higher in comparison to the level of auxin activity present in the medium, which may typically be a cytokinin:auxin ratio of at least about 1:1 or higher in terms of weight/volume provided. The exact cytokinin:auxin ratio, however, will depend on the exact chemical identities of the auxin and cytokinin since different auxins and cytokinins can have different activities and/or modes of action, as known in the art. The levels of cytokinin and auxin in a medium having a high cytokinin to auxin ratio may be present in the medium (measured in terms of weight/volume), for example, at a ratio of about 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1, including all ranges derivable therebetween.

The levels of cytokinin and auxin in a culture medium having a high cytokinin to auxin ratio may be, for example, greater than or equal to about 1:1 or at least about 1:1 or higher, greater than or equal to about 1.5:1 or at least about 1.5:1 or higher, greater than or equal to about 2:1 or at least about 2:1 or higher, greater than or equal to about 2.5:1 or at least about 2.5:1 or higher, greater than or equal to about 3:1 or at least about 3:1 or higher, greater than or equal to about 3.5:1 or at least about 3.5:1 or higher, greater than or equal to about 4:1 or at least about 4:1 or higher, greater than or equal to about 4.5:1 or at least about 4.5:1 or higher, greater than or equal to about 5:1 or at least about 5:1 or higher, greater than or equal to about 5.5:1 or at least about 5.5:1 or higher, greater than or equal to about 6:1 or at least about 6:1 or higher, greater than or equal to about 6.5:1 or at least about 6.5:1 or higher, greater than or equal to about 7:1 or at least about 7:1 or higher, greater than or equal to about 7.5:1 or at least about 7.5:1 or higher, greater than or equal to about 8:1 or at least about 8:1 or higher, greater than or equal to about 8.5:1 or at least about 8.5:1 or higher, greater than or equal to about 9:1 or at least about 9:1 or higher, greater than or equal to about 9.5:1 or at least about 9.5:1 or higher, or about 10:1, including all ranges derivable therebetween.

The levels of cytokinin and auxin in a culture medium having a high cytokinin to auxin ratio may be, for example, in a range between about 1:1 and about 10:1, about 1.5:1 and about 10:1, about 2:1 and about 10:1, about 2.5:1 and about 10:1, about 3:1 and about 10:1, about 3.5:1 and about 10:1, about 4:1 and about 10:1, about 4.5:1 and about 10:1, about 5:1 and about 10:1, about 5.5:1 and about 10:1, about 6:1 and about 10:1, about 6.5:1 and about 10:1, about 7:1 and about 10:1, about 7.5:1 and about 10:1, about 8:1 and about 10:1, about 8.5:1 and about 10:1, about 9:1 and about 10:1, or about 9.5:1 and about 10:1, including all ranges derivable therebetween.

In some embodiments, the concentration of the cytokinin in the germination medium is in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.1 mg/L to about 1.0 mg/L, about 0.1 mg/L to about 0.75 mg/L, about 0.1 mg/L to about 0.7 mg/L, about 0.1 mg/L to about 0.6 mg/L, about 0.2 mg/L to about 10.0 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.2 mg/L to about 1.0 mg/L, about 0.2 mg/L to about 0.75 mg/L, about 0.2 mg/L to about 0.7 mg/L, about 0.2 mg/L to about 0.6 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.5 mg/L to about 1.0 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.75 mg/L to about 1.0 mg/L, about 1.0 mg/L to about 5.0 mg/L, about 1.0 mg/L to about 4.0 mg/L, about 1.0 mg/L to about 3.0 mg/L, about 1.5 mg/L to about 2.5 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.8 mg/L to about 1.0 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, about 0.9 mg/L to about 1.0 mg/L, about 0.3 mg/L to about 1.0 mg/L, about 0.3 mg/L to about 0.75 mg/L, about 0.3 mg/L to about 0.7 mg/L, about 0.3 mg/L to about 0.6 mg/L, about 0.4 mg/L to about 1.0 mg/L, about 0.4 mg/L to about 0.75 mg/L, about 0.4 mg/L to about 0.7 mg/L, about 0.4 mg/L to about 0.6 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 10.0 mg/L, about 4.0 mg/L to about 7.5 mg/L, about 4.0 mg/L to about 7.0 mg/L, or about 4.0 mg/L to about 6.0 mg/L, including all ranges derivable therebetween.

In some embodiments, the concentration of the cytokinin in the germination medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 5.5 mg/L, about 6.0 mg/L, about 6.5 mg/L, about 7.0 mg/L, about 7.5 mg/L, about 8.0 mg/L, about 8.5 mg/L, about 9.0 mg/L, about 9.5 mg/L, or about 10.0 mg/L, including all concentrations derivable therebetween.

In some embodiments, the concentration of the auxin in the germination medium is in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.1 mg/L to about 1.0 mg/L, about 0.1 mg/L to about 0.75 mg/L, about 0.1 mg/L to about 0.7 mg/L, about 0.1 mg/L to about 0.6 mg/L, about 0.2 mg/L to about 10.0 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.2 mg/L to about 1.0 mg/L, about 0.2 mg/L to about 0.75 mg/L, about 0.2 mg/L to about 0.7 mg/L, about 0.2 mg/L to about 0.6 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.5 mg/L to about 1.0 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.75 mg/L to about 1.0 mg/L, about 1.0 mg/L to about 5.0 mg/L, about 1.0 mg/L to about 4.0 mg/L, about 1.0 mg/L to about 3.0 mg/L, about 1.5 mg/L to about 2.5 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.8 mg/L to about 1.0 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, about 0.9 mg/L to about 1.0 mg/L, about 0.3 mg/L to about 1.0 mg/L, about 0.3 mg/L to about 0.75 mg/L, about 0.3 mg/L to about 0.7 mg/L, about 0.3 mg/L to about 0.6 mg/L, about 0.4 mg/L to about 1.0 mg/L, about 0.4 mg/L to about 0.75 mg/L, about 0.4 mg/L to about 0.7 mg/L, about 0.4 mg/L to about 0.6 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 10.0 mg/L, about 4.0 mg/L to about 7.5 mg/L, about 4.0 mg/L to about 7.0 mg/L, or about 4.0 mg/L to about 6.0 mg/L, including all ranges derivable therebetween.

In some embodiments, the concentration of the auxin in the germination medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 5.5 mg/L, about 6.0 mg/L, about 6.5 mg/L, about 7.0 mg/L, about 7.5 mg/L, about 8.0 mg/L, about 8.5 mg/L, about 9.0 mg/L, about 9.5 mg/L, or about 10.0 mg/L, including all concentrations derivable therebetween.

In some embodiments, the concentration of the gibberellin in the germination medium is in the range from about 0.01 mg/L to about 10.0 mg/L, about 0.05 mg/L to about 10.0 mg/L, about 0.01 mg/L to about 7.5 mg/L, about 0.05 mg/L to about 7.5 mg/L, about 0.01 mg/L to about 5.0 mg/L, about 0.05 mg/L to about 5.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.1 mg/L to about 1.0 mg/L, about 0.1 mg/L to about 0.75 mg/L, about 0.1 mg/L to about 0.7 mg/L, about 0.1 mg/L to about 0.6 mg/L, about 0.2 mg/L to about 10.0 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.2 mg/L to about 1.0 mg/L, about 0.2 mg/L to about 0.75 mg/L, about 0.2 mg/L to about 0.7 mg/L, about 0.2 mg/L to about 0.6 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.5 mg/L to about 1.0 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.75 mg/L to about 1.0 mg/L, about 1.0 mg/L to about 5.0 mg/L, about 1.0 mg/L to about 4.0 mg/L, about 1.0 mg/L to about 3.0 mg/L, about 1.5 mg/L to about 2.5 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.8 mg/L to about 1.0 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, about 0.9 mg/L to about 1.0 mg/L, about 0.3 mg/L to about 1.0 mg/L, about 0.3 mg/L to about 0.75 mg/L, about 0.3 mg/L to about 0.7 mg/L, about 0.3 mg/L to about 0.6 mg/L, about 0.4 mg/L to about 1.0 mg/L, about 0.4 mg/L to about 0.75 mg/L, about 0.4 mg/L to about 0.7 mg/L, about 0.4 mg/L to about 0.6 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 10.0 mg/L, about 4.0 mg/L to about 7.5 mg/L, about 4.0 mg/L to about 7.0 mg/L, or about 4.0 mg/L to about 6.0 mg/L, including all ranges derivable therebetween.

In some embodiments, the concentration of the gibberellin in the germination medium may be, for example, about 0.01 mg/L, about 0.02 mg/L, about 0.03 mg/L, about 0.04 mg/L, about 0.05 mg/L, about 0.06 mg/L, about 0.07 mg/L, about 0.08 mg/L, about 0.09 mg/L, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 5.5 mg/L, about 6.0 mg/L, about 6.5 mg/L, about 7.0 mg/L, about 7.5 mg/L, about 8.0 mg/L, about 8.5 mg/L, about 9.0 mg/L, about 9.5 mg/L, or about 10.0 mg/L, including all concentrations derivable therebetween.

In some embodiments, the concentration of activated charcoal is present in said germination medium at a concentration of from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.1 mg/L to about 1.0 mg/L, about 0.1 mg/L to about 0.75 mg/L, about 0.1 mg/L to about 0.7 mg/L, about 0.1 mg/L to about 0.6 mg/L, about 0.2 mg/L to about 10.0 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.2 mg/L to about 1.0 mg/L, about 0.2 mg/L to about 0.75 mg/L, about 0.2 mg/L to about 0.7 mg/L, about 0.2 mg/L to about 0.6 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.5 mg/L to about 1.0 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.75 mg/L to about 1.0 mg/L, about 1.0 mg/L to about 5.0 mg/L, about 1.0 mg/L to about 4.0 mg/L, about 1.0 mg/L to about 3.0 mg/L, about 1.5 mg/L to about 2.5 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.8 mg/L to about 1.0 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, about 0.9 mg/L to about 1.0 mg/L, about 0.3 mg/L to about 1.0 mg/L, about 0.3 mg/L to about 0.75 mg/L, about 0.3 mg/L to about 0.7 mg/L, about 0.3 mg/L to about 0.6 mg/L, about 0.4 mg/L to about 1.0 mg/L, about 0.4 mg/L to about 0.75 mg/L, about 0.4 mg/L to about 0.7 mg/L, about 0.4 mg/L to about 0.6 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 10.0 mg/L, about 4.0 mg/L to about 7.5 mg/L, about 4.0 mg/L to about 7.0 mg/L, or about 4.0 mg/L to about 6.0 mg/L, including all ranges derivable therebetween.

In some embodiments, the concentration of the activated charcoal in the germination medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 5.5 mg/L, about 6.0 mg/L, about 6.5 mg/L, about 7.0 mg/L, about 7.5 mg/L, about 8.0 mg/L, about 8.5 mg/L, about 9.0 mg/L, about 9.5 mg/L, or about 10.0 mg/L, including all concentrations derivable therebetween.

In some embodiments, the concentration of polyvinylpyrrolidone is present in said germination medium at a concentration of about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.5 mg/L to about 1.0 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.75 mg/L to about 1.0 mg/L, about 1.0 mg/L to about 5.0 mg/L, about 1.0 mg/L to about 4.0 mg/L, about 1.0 mg/L to about 3.0 mg/L, about 1.5 mg/L to about 2.5 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.8 mg/L to about 1.0 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, about 0.9 mg/L to about 1.0 mg/L, about 0.3 mg/L to about 1.0 mg/L, about 0.3 mg/L to about 0.75 mg/L, about 0.3 mg/L to about 0.7 mg/L, about 0.3 mg/L to about 0.6 mg/L, about 0.4 mg/L to about 1.0 mg/L, about 0.4 mg/L to about 0.75 mg/L, about 0.4 mg/L to about 0.7 mg/L, or about 0.4 mg/L to about 0.6 mg/L, including all ranges derivable therebetween.

In some embodiments, the concentration of the polyvinylpyrrolidone in the germination medium may be, for example, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, or about 5.0 mg/L, including all concentrations derivable therebetween.

In some embodiments, the concentration of leucine is present in said germination medium at a concentration of about 1.2 mg/L to about 120.0 mg/L, about 1.2 mg/L to about 110.0 mg/L, about 1.2 mg/L to about 100.0 mg/L, about 1.2 mg/L to about 90.0 mg/L, about 1.2 mg/L to about 80.0 mg/L, about 1.2 mg/L to about 75.0 mg/L, about 2.5 mg/L to about 120.0 mg/L, about 2.5 mg/L to about 110.0 mg/L, about 2.5 mg/L to about 100.0 mg/L, about 2.5 mg/L to about 90.0 mg/L, about 2.5 mg/L to about 80.0 mg/L, about 2.5 mg/L to about 75.0 mg/L, about 5.0 mg/L to about 120.0 mg/L, about 5.0 mg/L to about 110.0 mg/L, about 5.0 mg/L to about 100.0 mg/L, about 5.0 mg/L to about 90.0 mg/L, about 5.0 mg/L to about 80.0 mg/L, about 5.0 mg/L to about 75.0 mg/L, about 5.0 mg/L to about 70.0 mg/L, about 7.5 mg/L to about 120.0 mg/L, about 7.5 mg/L to about 110.0 mg/L, about 7.5 mg/L to about 100.0 mg/L, about 7.5 mg/L to about 90.0 mg/L, about 7.5 mg/L to about 80.0 mg/L, about 7.5 mg/L to about 75.0 mg/L, about 10.0 mg/L to about 120.0 mg/L, about 10.0 mg/L to about 110.0 mg/L, about 10.0 mg/L to about 100.0 mg/L, about 10.0 mg/L to about 90.0 mg/L, about 10.0 mg/L to about 80.0 mg/L, about 10.0 mg/L to about 75.0 mg/L, about 10.0 mg/L to about 70.0 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, or about 10.0 mg/L to about 15.0 mg/L, including all ranges derivable therebetween.

In some embodiments, the concentration of leucine is present in said germination medium at a concentration of about 1.2 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, about 25.0 mg/L, about 30 mg/L, about 40 mg/L, about 50 mg/L, about 60 mg/L, about 70 mg/L, about 75 mg/L, about 80 mg/L, about 90 mg/L, about 100 mg/L, about 110 mg/L, or about 120 mg/L, including all concentrations derivable therebetween.

In some embodiments, the concentration of spermidine is present in said germination medium at a concentration of about 1.4 mg/L to about 140.0 mg/L, about 1.4 mg/L to about 130.0 mg/L, about 1.4 mg/L to about 120.0 mg/L, about 1.4 mg/L to about 110.0 mg/L, about 1.4 mg/L to about 100.0 mg/L, about 1.4 mg/L to about 90.0 mg/L, about 1.4 mg/L to about 80.0 mg/L, about 1.4 mg/L to about 75.0 mg/L, about 2.5 mg/L to about 140.0 mg/L, about 2.5 mg/L to about 130.0 mg/L, about 2.5 mg/L to about 120.0 mg/L, about 2.5 mg/L to about 110.0 mg/L, about 2.5 mg/L to about 100.0 mg/L, about 2.5 mg/L to about 90.0 mg/L, about 2.5 mg/L to about 80.0 mg/L, about 2.5 mg/L to about 75.0 mg/L, about 5.0 mg/L to about 140.0 mg/L, about 5.0 mg/L to about 130.0 mg/L, about 5.0 mg/L to about 120.0 mg/L, about 5.0 mg/L to about 110.0 mg/L, about 5.0 mg/L to about 100.0 mg/L, about 5.0 mg/L to about 90.0 mg/L, about 5.0 mg/L to about 80.0 mg/L, about 5.0 mg/L to about 75.0 mg/L, about 5.0 mg/L to about 70.0 mg/L, about 7.5 mg/L to about 140.0 mg/L, about 7.5 mg/L to about 130.0 mg/L, about 7.5 mg/L to about 120.0 mg/L, about 7.5 mg/L to about 110.0 mg/L, about 7.5 mg/L to about 100.0 mg/L, about 7.5 mg/L to about 90.0 mg/L, about 7.5 mg/L to about 80.0 mg/L, about 7.5 mg/L to about 75.0 mg/L, about 10.0 mg/L to about 140.0 mg/L, about 10.0 mg/L to about 130.0 mg/L, about 10.0 mg/L to about 120.0 mg/L, about 10.0 mg/L to about 110.0 mg/L, about 10.0 mg/L to about 100.0 mg/L, about 10.0 mg/L to about 90.0 mg/L, about 10.0 mg/L to about 80.0 mg/L, about 10.0 mg/L to about 75.0 mg/L, about 10.0 mg/L to about 70.0 mg/L, about 2.0 mg/L to about 50.0 mg/L, about 2.0 mg/L to about 25.0 mg/L, about 2.0 mg/L to about 20.0 mg/L, about 2.0 mg/L to about 15.0 mg/L, about 5.0 mg/L to about 50.0 mg/L, about 5.0 mg/L to about 25.0 mg/L, about 5.0 mg/L to about 20.0 mg/L, about 5.0 mg/L to about 15.0 mg/L, about 7.5 mg/L to about 50.0 mg/L, about 7.5 mg/L to about 25.0 mg/L, about 7.5 mg/L to about 20.0 mg/L, about 7.5 mg/L to about 15.0 mg/L, or about 10.0 mg/L to about 15.0 mg/L, including all ranges derivable therebetween.

In some embodiments, the concentration of spermidine is present in said germination medium at a concentration of about 1.4 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, about 25.0 mg/L, about 30.0 mg/L, about 40.0 mg/L, about 50.0 mg/L, about 60.0 mg/L, about 70.0 mg/L, about 75.0 mg/L, about 80.0 mg/L, about 90.0 mg/L, about 100.0 mg/L, about 110.0 mg/L, about 120.0 mg/L, about 130.0 mg/L, or about 140.0 mg/L, including all concentrations derivable therebetween.

In some embodiments, the concentration of pyridoxine HCl is present in said germination medium at a concentration of about 0.05 mg/L to about 5.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.0 mg/L, about 0.1 mg/L to about 0.75 mg/L, about 0.1 mg/L to about 0.7 mg/L, about 0.1 mg/L to about 0.6 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.0 mg/L, about 0.2 mg/L to about 0.75 mg/L, about 0.2 mg/L to about 0.7 mg/L, about 0.2 mg/L to about 0.6 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.5 mg/L to about 1.0 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.75 mg/L to about 1.0 mg/L, about 1.0 mg/L to about 5.0 mg/L, about 1.0 mg/L to about 4.5 mg/L, about 1.0 mg/L to about 4.0 mg/L, about 1.0 mg/L to about 3.5 mg/L, about 1.0 mg/L to about 3.0 mg/L, about 1.5 mg/L to about 5.0 mg/L, about 1.5 mg/L to about 4.5 mg/L, about 1.5 mg/L to about 4.0 mg/L, about 1.0 mg/L to about 5.0 mg/L, about 1.0 mg/L to about 4.5 mg/L, about 1.0 mg/L to about 4.0 mg/L, about 1.0 mg/L to about 3.5 mg/L, about 1.0 mg/L to about 3.0 mg/L, about 0.75 mg/L to about 4.0 mg/L, about 0.75 mg/L to about 3.5 mg/L, about 0.75 mg/L to about 3.0 mg/L, about 0.75 mg/L to about 2.5 mg/L, about 0.75 mg/L to about 2.0 mg/L, about 0.25 mg/L to about 5.0 mg/L, about 0.25 mg/L to about 4.5 mg/L, about 0.25 mg/L to about 4.0 mg/L, about 0.25 mg/L to about 3.5 mg/L, about 0.25 mg/L to about 3.0 mg/L, about 0.25 mg/L to about 2.0 mg/L, about 0.25 mg/L to about 1.0 mg/L, about 0.075 mg/L to about 5.0 mg/L, including all ranges derivable therebetween.

In some embodiments, the concentration of the pyridoxine HCl present in the germination medium may be, for example, about 0.05 mg/L, about 0.06 mg/L, about 0.07 mg/L, about 0.08 mg/L, about 0.09 mg/L, about 0.1 mg/L, about 0.2 mg/L, about 0.25 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, or about 5.0 mg/L, including all concentrations derivable therebetween.

As used herein, “pepper microspore salt and vitamin mixture” refers to a solution of specific macro elements, micro elements, vitamins, amino acids, and chelated iron, all present at specific concentrations, that was developed by the present inventors for use in the methods described herein. The salt and vitamin mixture may be used as a base nutrient medium or as a supplement to a standard tissue culture medium.

As used herein, “MS3 plantlet development medium” refers specifically to a culture medium that is used for further plantlet development. The MS3 plantlet development medium described herein comprises MS basal salts, MS vitamins, polyvinylpyrrolidone, plant agar, and an organic carbon source, preferably sucrose.

As used herein, “GPG medium” refers to a solid medium that is used for further plant development and acclimatization. The GPG medium for use with the present invention contains the pepper microspore salt and vitamin mixture, MES buffer, Ca(NO₃)₂, Gelrite, and an organic carbon source, preferably sucrose.

During in vitro culture, the plant tissue can turn brown due to the release and accumulation of phenolic compounds. Browning can have negative effects such as poor embryo growth or death. Addition of polyvinylpyrrolidone (PVP) to a number of media used throughout the process prevented browning of the embryo and helped improve germination. As indicated above, PVP is a component of the washing, culturing, germination, and plantlet development media developed for the present invention.

Successful in vitro cell culture depends on proper cell density at the onset of the culture. Previous protocols have emphasized the importance of microspore density after they have been isolated and prior to initiation of culture: densities that are too low can result in no observable embryogenic response; however, microspore densities that are too high may inhibit the embryogenic response and/or reduce the number of viable embryos due to the reduced availability of nutrients and/or to the presence of inhibitory toxins generated by the microspores. The present inventors have identified multiple points during the process of microspore cultivation and the optimal concentrations of microspores at these points for optimal efficiency. First, it was determined that during the microspore isolation and induction step, the microspores should be present in a stress medium of at a density of about 6.0×10⁴ cells/mL to about 10.0×10⁴ cells/mL, preferably at a density of about 6.80×10⁴ cells/mL to about 9.20×10⁴ cells/mL. Second, during the microspore liquid culture step, the microspores should be present in liquid NLN medium at a density of about 3.75×10⁴ cells/mL to about 6.25'10⁴ cells/mL, preferably at a density of about 4.25×10⁴ cells/mL to about 5.75×10⁴ cells/mL. Lastly, the double layer culture step should be carried out at a density of about 1.125×10⁴ cells/mL to about 1.875×10⁴ cells/mL, preferably at a density of about 1.28×10⁴ cells/mL to about 1.73×10⁴ cells/mL, in a culture dish containing solid NLN medium. It is understood that the microspore culture density may be adjusted based on the species being cultured.

Microspore-derived haploid embryos can be converted to doubled haploids by chromosome doubling agents and/or through spontaneous doubling. Chromosome doubling agents are known in the art and include colchicine, amiprophos-methyl, oryzalin, pronamide, trifluralin, etc. As used herein, when referring to chromosome count, “doubling” refers to increasing the chromosome number by a factor of two. For example, a haploid nuclear genome comprising 10 chromosomes is doubled to become a diploid nuclear genome comprising 20 chromosomes. As another example, a diploid nuclear genome comprising 20 chromosomes is doubled to become a tetraploid nuclear genome comprising 40 chromosomes. Confirmation of chromosome doubling can be carried out by flow cytometry or other molecular biology techniques known in the art.

### Definitions

An “allele” refers to one or more alternative forms of a genetic sequence; the length of an allele can be as small as 1 nucleotide base. It can also refer to the absence of a sequence. For example, a first allele can occur on one chromosome, while a second allele occurs on the homologous position of a second chromosome, e.g., as occurs for different chromosomes of a heterozygous individual, or between different homozygous or heterozygous individuals in a population. A favorable allele is the allele at a particular locus that confers, or contributes to, an agronomically desirable phenotype, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying disease prone plants. A favorable allelic form of a chromosome site or segment is a chromosome site or segment that includes a nucleotide sequence that contributes to superior agronomic performance at one or more genetic loci physically located on the chromosome interval. “Allele frequency” refers to the frequency (proportion or percentage) at which an allele is present at a locus within an individual, within a line, or within a population of lines. For example, for an allele “A,” diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele. An allele positively correlates with a trait when it is linked to it and when presence of the allele is an indicator that the desired trait or trait form will occur in a plant comprising the allele. An allele negatively correlates with a trait when it is linked to it and when presence of the allele is an indicator that a desired trait or trait form will not occur in a plant comprising the allele.

“Anther culture” is the process of culturing intact anthers.

“Crossed” or “cross” means to produce progeny via fertilization (e.g. cells, embryos, seeds or plants) and includes crosses between plants (sexual) and self-fertilization (selfing).

“Potentiated microspores” refer to those microspores that deviate from the normal microspore maturation process of becoming mature pollen. They adopt a different cell fate and have potential to differentiate into embryos. They often comprise doubled chromosomes but have not undergone cytokinesis. Potentiated microspores may be enriched or obtained by staging and/or specific treatments such as chemical (colchicine), various stresses (heat or cold), etc.

A “doubled haploid or doubled haploid plant or cell”, also referred to as a dihaploid or dihaploid plant or cell, is one that is developed by the doubling of a haploid set of chromosomes. A plant or seed that is obtained from a doubled haploid plant that is selfed any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes. For example, a plant will be considered a doubled haploid plant if it contains viable gametes, even if it is chimeric.

A “microspore-derived embryo” is an embryo that was derived from microspore through tissue culture.

A “doubled microspore-derived embryo” is a microspore-derived embryo that contains 2 sets of homozygous chromosomes.

“Doubled microspore-derived embryogenesis” is a measurement that takes into account of both embryogenesis efficiency and chromosome doubling efficiency. It is calculated by multiplying the total embryos derived from certain number of microspores by the chromosome doubling rate. For example, if there are 2000 embryos derived from 1 million microspores and the chromosome doubling rate is 90%, the doubled microspore-derived embryogenesis is 1800 embryos/million micro spores.

“Genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic makeup for all the genes in its genome, or its entire genetic makeup. The terms “phenotype,” or “phenotypic trait” or “trait” refers to one or more traits of an organism. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait.” In other cases, a phenotype is the result of the expression of several genes and their interaction with environments.

As used herein, the term “plurality” refers to more than one. Thus, a “plurality of individuals” refers to at least two individuals. In some embodiments, the term plurality refers to more than half of the whole. For example, in some embodiments a “plurality of a population” refers to more than half the members of that population.

### EXAMPLES

The following examples are included to illustrate embodiments of the invention. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Development of a Novel Method for Pepper Microspore Culture

This example describes a novel protocol for culture of pepper microspores for the production of doubled haploid plants, including the steps of sterilization of pepper buds, microspore isolation from sterilized buds, liquid culture of microspores, double-layer culture, embryo harvest, and acclimatization of cultured plantlets.

### Step 1—Sterilization of Pepper Flower Buds

Prior to sterilization, the staging process is carried out, which is a correlation between the nuclear stage of the microspores and the size of the buds. The nuclear stage is determined through a microscopy analysis, by staining the cells with DAPI (4′,6-diamidino-2-phenylindole). The development of the stained microspores is then classified as uninucleate or binucleate. The target stage is a mix of 70% uninucleate and 30% binucleate cells. After the microscopy visualization the nuclear stage is correlated with the bud size, and only buds presenting the target size are used for isolation. After flower buds are obtained from donor pepper plants and are sterilized by immersing the buds in a sterilizing solution comprising 2% sodium hypochlorite with 0.01% Tween 20. The flower buds are soaked in the sterilizing solution for approximately 14 minutes. Next, the sterilizing solution is removed, and the buds are washed 3 times with sterile water

### Step 2—Microspore Isolation

A transversal cut is made approximately 1-2 mm above the bottom part of the bud. The ovary is removed, and the remainder of the bud is placed in a sterilized electric grinder for 30 seconds at 2500 rpm. The microspore suspension is filtered through a 70-100 μm cell strainer. The grinder is rinsed with 25 mL of washing medium and the suspension is filtered through the cell strainer. Prior to its use, the washing medium was stored at 4° C. The composition of the washing medium is shown in Table 1. The microspore suspension is filtered again using a 40 μm cell strainer.

TABLE 1
Composition of washing medium.

| Component | Concentration | |
|---|---|---|
| B5 medium, including vitamins | 3.163 | g/L |
| Sucrose | 130.0 | g/L |
| Polyvinylpyrrolidone | 1.0 | g/L |
| Cefotaxime | 100.0 | g/L |

Immediately after grinding and filtering, the microspore suspension must be centrifuged, preferably at 100 g for 5 minutes at 8° C. The supernatant is removed and the microspore pellet is resuspended in 40 mL of washing medium. The suspension is centrifuged at 100 g for 5 minutes at 8° C. The supernatant is removed, and the microspore pellet is resuspended in induction medium at a density of about 6.80×10⁴ cells/mL to about 9.20×10⁴ cells/mL, preferably about 8.0×10⁴ cells/mL. Prior to its use, the inducation medium was stored at 4° C. The composition of the induction medium is shown in Table 2. Following resuspension in induction medium, 1 μM of Trichostatin A is added to the microspore solution. Aliquots of 15-20 mL of the microspore solution are distributed in 95×25 mm Petri dishes and incubated at 32±1° C. for about 24 hours to about 96 hours (preferably 72 hours) to induce embryogenesis. The plates containing the microspore culture were sealed with one to two layers of film.

TABLE 2
Composition of induction medium.

| Component | Concentration | |
|---|---|---|
| NLN salt mixture | 0.386 | g/L |
| NLN vitamin mixture | 1.038 | g/L |
| NLN stock solution | 10.0 | ml/L |
| Mannitol | 67.0 | g/L |
| Gum arabic | 10.0 | mg/L |
| Cefotaxime | 100.0 | mg/L |

### Step 3—Liquid Culture

After induction treatment, the cell suspension is centrifuged at 300 g for 5 minutes at 15° C. The supernatant is discarded and the pellet containing the microspores is resuspended in NLN liquid medium to a density of about 4.25×10⁴ cells/mL to about 5.75×10⁴ cells/mL, preferably about 5.0×10⁴ cells/mL, in 60×15 mm Petri dishes and incubated at 27° C. for one week for progression of embryogenesis. Prior to its use, the NLN liquid medium was stored at 4° C. The composition of the NLN liquid medium is shown in Table 3 below.

TABLE 3
Composition of NLN liquid medium.

| Medium Component | Concentration | |
|---|---|---|
| NLN salt mixture | 0.386 | g/L |
| NLN vitamin mixture | 1.038 | g/L |
| NLN stock solution | 10.0 | ml/L |
| Sucrose | 90.0 | g/L |
| Gum arabic | 10.0 | mg/L |
| Cefotaxime | 100.0 | mg/L |
| Polyvinylpyrrolidone | 1.0 | g/L |

### Step 4—Double-Layer Culture

The suspension from Step 3 above is resuspended in fresh NLN liquid medium to a final density of about 1.28×10⁴ cells/mL to about 1.73×10⁴ cells/mL, preferably about 1.5×10⁴ cells/mL, and cultured on a double-layer medium. The double-layer medium culture method consists of the microspore/NLN liquid medium as the upper layer atop a NLN solid under layer in a 95×25 mm Petri dish. The culture is incubated at 27° C. for 30 days. The composition of the NLN medium solid layer is shown in Table 4.

TABLE 4
Composition of NLN solid medium.

| Component | Concentration | |
|---|---|---|
| NLN salt mixture | 0.386 | g/L |
| NLN vitamin mixture | 1.038 | g/L |
| NLN stock solution | 10.0 | ml/L |
| Sucrose | 20.0 | g/L |
| Gum arabic | 10.0 | mg/L |
| Cefotaxime | 100.0 | mg/L |
| Polyvinylpyrrolidone | 1.0 | g/L |
| Phytagel ™ | 4.0 | g/L |

### Step 5—Embryo Harvest

After 4 weeks in the double-layer culture, the embryos are harvested by transferring the liquid phase into a clean tube. Next, the expend medium is removed and replaced with sterile water. The embryo suspension is placed on top of at least one filter paper (preferably two or three filter papers) that is on top of 25 mL of solid germination medium, preferably MSPG12, shown in Table 5 below.

TABLE 5
Composition of MSPG1, MSPG3, MSPG5, MSPG7,
and MSPG12 germination mediums.

| | MSPG1 | MSPG3 | MSPG5 | MSPG7 | MSPG12 |
|---|---|---|---|---|---|
| Component | Mg/L | | | | |
| CaCl₂ | 166.01 | 166.01 | 166.01 | 166.01 | 166.01 |
| MgSO₄ | 90.27 | 90.27 | 90.27 | 90.27 | 90.27 |
| NH₄NO₃ | 825.0 | 825.0 | 825.0 | 825.0 | 1444.0 |
| KH₂PO₄ | 85.0 | 85.0 | 85.0 | 85.0 | 156.0 |
| KNO₃ | 950.0 | 950.0 | 950.0 | 950.0 | 2025.0 |
| MnSO₄•H₂O | 8.45 | 8.45 | 8.45 | 8.45 | 18.515 |
| ZnSO₄•7H₂O | 4.3 | 4.3 | 4.3 | 4.3 | 5.9125 |
| H₃BO₃ | 3.1 | 3.1 | 3.1 | 3.1 | 3.875 |
| KI | 0.415 | 0.415 | 0.415 | 0.415 | 0.58 |
| Na₂MoO₄•2H₂O | 0.125 | 0.125 | 0.125 | 0.125 | 0.194 |
| CuSO₄•5H₂O | 0.0125 | 0.0125 | 0.0125 | 0.0125 | 0.018 |
| CoCl₂•6H₂O | 0.0125 | 0.0125 | 0.0125 | 0.0125 | 0.018 |
| Myo-Inositol | 50.0 | 50.0 | 50.0 | 50.0 | 75.15 |
| Pyridoxine HCl | 0.25 | 0.25 | 0.25 | 0.25 | 3.0 |
| Nicotinic Acid | 0.25 | 0.25 | 0.25 | 0.25 | 0.6 |
| Thiamine HCl | 0.05 | 0.05 | 0.05 | 0.05 | 0.35 |
| Glycine | 1.0 | 1.0 | 1.0 | 1.0 | 1.05 |
| Sucrose | 40.0 | 40.0 | 40.0 | 40.0 | 40.0 |
| FeNaEDTA | 18.35 | 18.35 | 18.35 | 18.35 | 34.6 |
| Plant Agar | 8.0 | 8.0 | 8.0 | 8.0 | 8.0 |
| Kinetin | 1.0 | 1.0 | 1.0 | — | — |
| Thidiazuron (TDZ) | — | — | — | 2.0 | 2.0 |
| Gibberellic Acid (GA) | 1.0 | 1.0 | 1.0 | 1.0 | 1.0 |
| 1-Naphthaleneacetic | 1.0 | — | — | — | — |
| Acid (NAA) | | | | | |
| Indole-3-acetic Acid | — | 1.0 | 1.0 | 0.5 | 0.5 |
| (IAA) | | | | | |
| Polyvinylpyrrolidone | | | 1.0 | 1.0 | 1.0 |
| Activated Charcoal | | | 1.0 | 1.0 | 1.0 |
| Leucine | | | 12.0 | 12.0 | 12.0 |
| Spermidine | | | 14.0 | 14.0 | 14.0 |
| CaCl₂•2H₂O | | | | | 156.5 |
| MgSO₄•7H₂O | | | | | 206.0 |
| NaH₂PO₄•H₂O | | | | | 19.0 |
| (NH₄)₂SO₄ | | | | | 17.0 |
| KCl | | | | | 3.5 |
| Calcium Pantothenate | | | | | 0.25 |
| Biotin | | | | | 0.05 |
| MES | | | | | 250.0 |
| Ca(NO₃)₂ | | | | | 25.0 |

The embryos are incubated at 26° C. in a 16 hour light/8 hour dark photoperiod. After 3 weeks and 6 weeks, the germinated embryos are transferred to MS3 medium for further plantlet development. Subsequently, the plantlets are transferred to GPG medium to grow at 18° C. The compositions of MS3 plantlet development medium and GPG medium are shown in Table 6 and Table 7, respectively.

TABLE 6
Composition of MS3 plantlet development medium.

| Component | Concentration (g/L) |
|---|---|
| MS medium with vitamins | 4.4 |
| Sucrose | 30.0 |
| Polyvinylpyrrolidone | 1.0 |
| Plant agar | 8.0 |

TABLE 7
Composition of GPG medium.

| Component | Concentration (g/L) |
|---|---|
| Pepper microspore salt and | 4.162 |
| vitamin mixture (see Table 8) | |
| Sucrose | 30.0 |
| MES | 0.5 |
| Ca(NO₃)₂ | 0.25 |
| Gelrite | 2.5 |

TABLE 8
Composition of pepper microspore salt and vitamin mixture.

| Component | Concentration (mg/L) |
|---|---|
| Macro Elements | |
| KNO₃ | 2150.0 |
| NH₄NO₃ | 1238.0 |
| MgSO₄•7H₂O | 412.0 |
| CaCl₂•2H₂O | 313.0 |
| KH₂PO₄ | 142.0 |
| NaH₂PO₄•H₂O | 38.0 |
| (NH₄)₂SO₄ | 34.0 |
| KCl | 7.0 |
| Micro Elements | |
| MnSO₄•H₂O | 20.13 |
| ZnSO₄•7H₂O | 3.225 |
| H₃BO₃ | 1.55 |
| KI | 0.33 |
| Na₂MoO₄•2H₂O | 0.138 |
| CuSO₄•5H₂O | 0.011 |
| CoCl₂•6H₂O | 0.011 |
| Vitamins and Amino Acids | |
| Myo-inositol | 50.3 |
| Pyridoxine HCl | 5.5 |
| Nicotinic acid | 0.7 |
| Thiamine HCl | 0.6 |
| Calcium pantothenate | 0.5 |
| Biotin | 0.005 |
| Glycine | 0.1 |
| Chelated Iron | |
| NaFeEDTA | 32.5 |

### Step 6 —Acclimatization of Cultured Plantlets

When roots are spread over the medium and 4-6 leaves have developed, the plantlets are ready to undergo acclimatization.

### Example 2: The New Germination Medium Significantly Improves Germination of Embryos

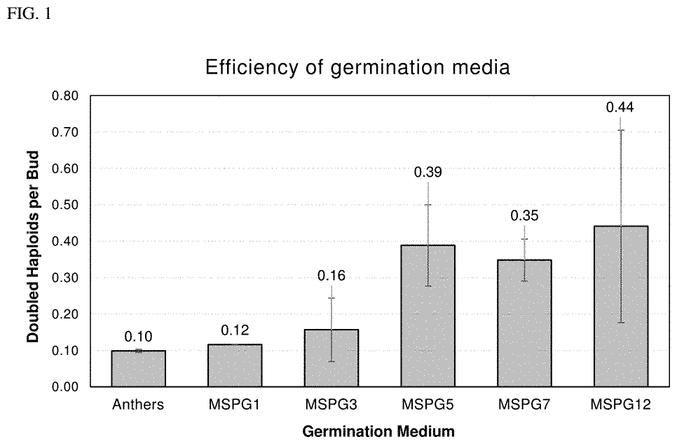

The germination medium described herein is required for functioning of the method. In the absence of this germination medium, most of the embryos are abnormal and do not develop properly. Different germination media compositions were tested until the MSPG12 formulation was identified as significantly improving the development of the embryos into plants, as shown in FIG. 1. The number of DHs obtained per bud when using an anther culture method was included as a reference comparison to assess the increase in efficiency. The formulation of this new germination medium required extensive testing and it resulted in a complex combination of many elements, amongst them a combination of: micronutrients, macronutrients, growth regulators, and other molecules like amino acids, vitamins, activated charcoal, polyamine, etc.

### Example 3: The New Microspore Method Significantly Improves the Number of Doubled Haploids Obtained per Bud

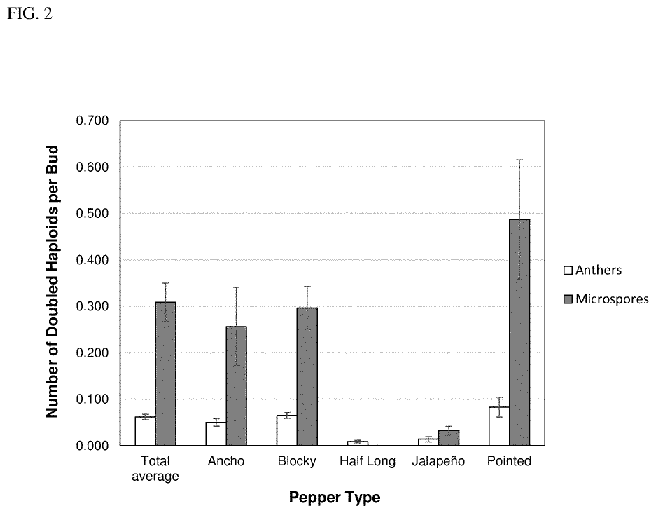

The method developed has a structure that is more complex than other microspore systems, however each of the steps has been demonstrated to be necessary for successful embryogenesis and subsequent germination and development of plants. The new microspore method was tested in 110 pepper origins, these origins can be classified in 5 types of pepper: Ancho, Blocky, Half Long, Jalapeño, and Pointed. Remarkably, the new microspore protocol works both for sweet and hot peppers (Ancho and Jalapeño). The ratio of DH obtained per bud is taken as a measure of efficiency. Employing the new microspore protocol improves the number of DHs expected by, on average, 5-fold (FIG. 2). Similarly, other ratios that measure efficiency, such as the number of embryos obtained per bud and the number of plants derived from a single bud also increase as shown in Table 9. Surprisingly, the new microspore method does not require supplementation with chromosome doubling agents, as the results show a high rate (57.9%) of spontaneous diploidization.

TABLE 9
Efficiency of anther culture method compared to the new microspore method.

| | # of | # of | # of | |
|---|---|---|---|---|
| | Embryos/Bud | Plants/Bud | DHs/bud | % Diploidization |
| Anthers | 0.38 ± 0.036 | 0.15 ± 0.014 | 0.062 ± 0.006 | 41.7% ± 1.59 |
| Microspores | 4.24 ± 0.64 | 0.59 ± 0.088 | 0.31 ± 0.041 | 57.9% ± 2.94 |

### Example 4: The Novel Microspore Method Improves Responsiveness in Recalcitrant Pepper Types

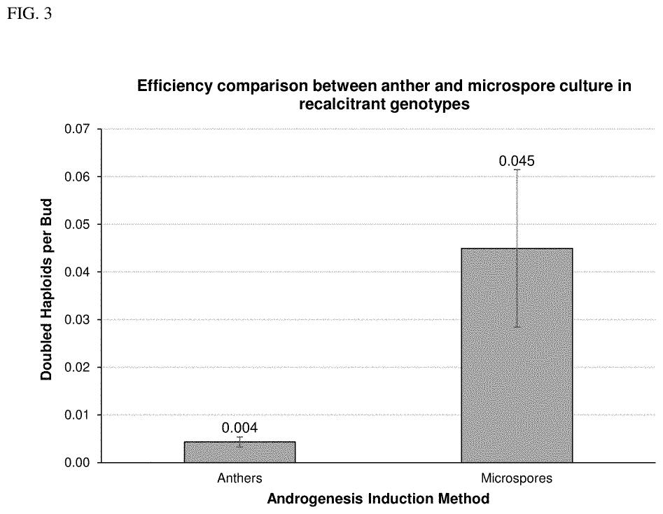

Recalcitrance is a phenomenon typical of in vitro culture, it is a described as a different response to the same method or protocol. Specifically, within a given species, different genotypes may be more or less responsive to a particular in vitro method. Recalcitrance has been observed in pepper anther culture. When using recalcitrant origins the highest efficiency obtained is of 0.01 doubled haploids per bud. Surprisingly, when testing the new method on the same 13 pepper origins that were recalcitrant to anther culture, there was a significant improvement (10-fold) in the responsiveness of recalcitrant material. By using the method described for the isolation and culturing of pepper microspores, viable embryos were obtained that were later developed to doubled haploid plants (FIG. 3).

### Example 5: The New Method Reduces the Workload and Total Time Required to Regenerate DH Plants

Employing the optimized method developed in this invention helps by reducing the total amount of time require for handling each bud. Specifically, the increase in the number of DHs obtained per bud also results in a reduction in the total time spent. These improved methods allow automatization and overall efficiency increase by reducing the handling times (Table 10).

TABLE 10
Workload comparison of employing conventional anther culture versus
the new microspore culture method for pepper DH production.

| | | Handling | | Approx. No. | |
|---|---|---|---|---|---|
| | | Time per | | of Buds | Hours to |
| | DHs/ | Single Bud | Target No. | Needed to | Target No. |
| | Bud | (sec) | of DHs | Reach Target | of DHs |
| Microspores | 0.31 | 170 | 100 | 322.60 | 15.23 |
| Anthers | 0.06 | 65 | 100 | 1,666.70 | 30.09 |

What is claimed is:

1. A method for producing embryos from microspores comprising the steps of:
a) isolating microspores from flower buds obtained from a donor pepper plant, wherein the microspores are at a developmental stage competent for induction of embryo development and wherein said microspores are isolated in a washing medium;
b) applying a stress treatment to said microspores comprised in an induction medium;
c) culturing said treated microspores in a culture medium;
d) subculturing said treated microspores as the liquid phase of a double layer culture medium; and
e) harvesting said embryos on a solid medium suitable for germination, wherein said medium comprises an auxin, a gibberellin, and a cytokinin, and wherein said cytokinin is present in said germination medium at a ratio of about 1:1 to about 10:1 relative to said auxin.

2. The method of claim 1, wherein said washing medium comprises an organic carbon source, wherein said organic carbon source is present in said medium at a high concentration.

3. The method of claim 1, wherein said stress treatment comprises incubating said microspores at a temperature of about 31° C. to about 33° C. for about 24 hours to about 96 hours.

4. The method of claim 1, wherein said stress treatment is carried out on microspores present in said medium at a density of about 6.0×10⁴ cells/mL to about 10.0×10⁴ cells/mL.

5. The method of claim 4, wherein said stress treatment is carried out on microspores present in said medium at a density of about 6.80×10⁴ cells/mL to about 9.20×10⁴ cells/mL.

6. The method of claim 1, wherein said culturing is carried out at a density of about 3.75×10⁴ cells/mL to about 6.25×10⁴ cells/mL.

7. The method of claim 6, wherein said culturing is carried out at a density of about 4.25×10⁴ cells/mL to about 5.75×10⁴ cells/mL.

8. The method of claim 1, wherein said subculturing is carried out at a density of about 1.125×10⁴ cells/mL to about 1.875×10⁴ cells/mL.

9. The method of claim 8, wherein said subculturing is carried out at a density of about 1.28×10⁴ cells/mL to about 1.73×10⁴ cells/mL.

10. The method of claim 1, wherein said auxin is selected from the group consisting of: 1-naphthaleneacetic acid, indole-3-acetic acid, and indole-3-butyric acid.

11. The method of claim 10, wherein said auxin is indole-3-acetic acid.

12. The method of claim 1, wherein said cytokinin is kinetin or thidiazuron.

13. The method of claim 12, wherein said cytokinin is thidiazuron.

14. The method of claim 1, wherein said gibberellin is gibberellic acid.

15. The method of claim 1, wherein said cytokinin to auxin ratio is from about 2:1 to about 5:1.

16. The method of claim 15, wherein said cytokinin to auxin ratio is about 4:1.

17. The method of claim 1, wherein said gibberellin is present in said germination medium at a concentration of about 0.01 mg/L to about 10 mg/L.

18. The method of claim 1, wherein said cytokinin is present in said germination medium at a concentration of about 0.1 mg/L to about 10 mg/L.

19. The method of claim 1, wherein said auxin is present in said germination medium at a concentration of about 0.1 mg/L to about 10 mg/L.

20. The method of claim 1, wherein said germination medium further comprises activated charcoal, polyvinylpyrrolidone, leucine, or spermidine.

21. The method of claim 20, wherein said activated charcoal is present in said germination medium at a concentration of about 0.1 mg/L to about 10 mg/L.

22. The method of claim 20, wherein said polyvinylpyrrolidone is present in said germination medium at a concentration of about 0.5 mg/L to about 5.0 mg/L.

23. The method of claim 20, wherein said leucine is present in said germination medium at a concentration of about 1.2 mg/L to about 120.0 mg/L.

24. The method of claim 20, wherein said spermidine is present in said germination medium at a concentration of about 1.4 mg/L to about 140.0 mg/L.

25. The method of claim 20, wherein said germination medium further comprises activated charcoal, polyvinylpyrrolidone, leucine, and spermidine.

26. The method of claim 1, wherein harvesting comprises harvesting the embryos on at least one filter paper on top of said solid medium.

27. The method of claim 1, wherein said germination medium comprises pyridoxine HCl present in said medium at a concentration of about 0.05 mg/L to about 5.0 mg/L.

28. The method of claim 2, wherein said organic carbon source is present in said washing medium at a concentration of about 50.0 g/L to about 200.0 g/L.

29. The method of claim 28, wherein said organic carbon source is present in said washing medium at a concentration of about 90.0 g/L to about 130.0 g/L.

30. The method of claim 2, wherein said organic carbon source is sucrose or maltose.

31. The method of claim 30, wherein said organic carbon source is sucrose.
